(19) United States
(12) Patent Application Publication
(10) Pub. No.: US 2010/0233823 A1
(43) Pub. Date: Sep. 16, 2010

(54) APPARATUS AND METHOD FOR OZONATION OF FLUIDS

(76) Inventor: JURGEN H. SCHEMMER, King City (CA)

Correspondence Address: WALTER A. HACKLER, PH.D.;PATENT LAW OFFICE, SUITE B, 2372S.E. BRISTOL STREET,, NEWPORT BEACH, CA, 92660-0755, US

(21) Appl. No.: 12/400,958

(22) Filed: Mar. 10, 2009

(51) Int. Cl. G01N 21/78; G01N 33/02; G01N 21/00

(52) U.S. Cl. 436/168; 422/68.1; 422/82.05

(57) ABSTRACT

Apparatus for ozonation of fluids includes a source of ozone, a fluid medium, and a container for receiving the fluid medium. Following charging of the fluid medium with ozone, an ozone sensor, which may be an ink, is utilized for determining the ozone content in the fluid medium within the container.

This invention relates to the modification of fluids with ozone. Oils, such as animal oils, including lard, meats foot and tallow oils, and fish oil, such as cod liver oil, salmon oil, and sardine oil; vegetable oils, such as corn, cottonseed, linseed, caster, and olive oils and mineral oils, for example, such as compositions of petroleum derivative and cod-liver oil may be modified to produce an edible or medicinal compound to retain ozone.

Gas combinations containing ozone also find many applications and the ozone content thereof is also vital to the application.

### SUMMARY OF THE INVENTION

Apparatus for the ozonation of fluids generally includes a source of ozone, a fluid medium, and a container for receiving the fluid medium.

Means are provided for charging a fluid medium ill the container with the ozone and further, means are provided for determining the ozone content in the ozonated fluid medium within the container.

More particularly, the means for determining ozone content comprises an ozone detecting ink which is disposed in the container. The means for charging the fluid medium may include a charging line connected to the source of ozone.

A method in accordance with the present invention for ozonation of the fluid generally includes providing a source of ozone along with a container and disposing the fluid in the container. Interconnection of the container with the source of ozone provides a transfer of ozone into the liquid and the amount of ozone in the fluid is determined through the use of an ozone detecting ink.

### BRIEF DESCRIPTION OF THE DRAWINGS

The advantages and features of the present invention will be better understood by the following description when considered in conjunction with the accompanying drawings in which:

FIG. 1 is an illustration of one embodiment of the present invention in which a container is utilized, the elements shown not being in perspective. A fluid contained in the vial is ozonated through the use of an ozone generator and an interconnecting line, also shown is an ozone detecting ink disposed in the container; and

FIG. 2 is a representation of yet another embodiment of the present invention in which all ozone generator is directly connected to a syringe with ozone detecting ink disposed in the syringe to determine ozone content.

### DETAILED DESCRIPTION

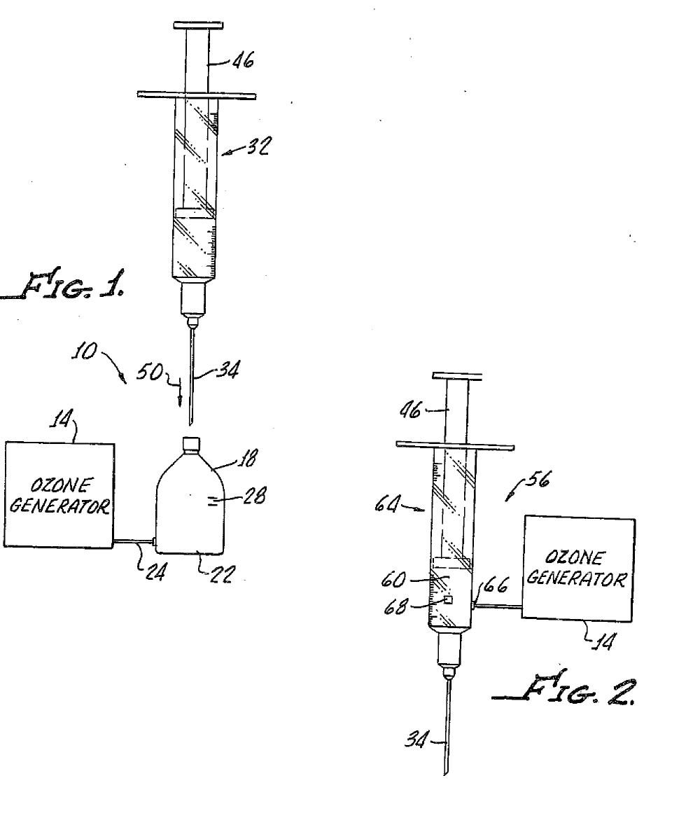

With reference to FIGS. 1 and 2, there is shown apparatus 10 in accordance with the present invention for ozonation of fluids.

The apparatus generally includes a source of ozone 14, which may be any suitable source, for example, as set forth in U.S. Pat. No. 7,270,544. This patent is to be incorporated herewith in its entirety for a complete description of an ozone generator 14 and system suitable for use in the present invention. A container 18, of any desirable size, 22, containing a fluid 22, and an interconnecting line 24 provides a means for ozonating the fluid.

Although a line 24 is shown interconnecting the ozone generator 14 and the container 18, any suitable charging mechanism may be utilized. An ozone detecting ink 28, or sensor, is provided inside the container 18 for determining the ozone content of the fluid 22 in the container 18. Such inks are described in U.S. Pat. No. 6,117,685 and this patent is incorporated herewith in its entirety to show all example of a means for detecting ozone content in the fluid 22.

Other color metric and system such as set forth in U.S. Pat. No. 3,397,966 and U.S. Pat. No. 4,859,607 may be utilized. These patents are also incorporated herewith in their entirety to show alternate means for determining ozone content in the aqueous solution 22. Preferably, any desirable ozone content may be charged, however, the ozone content should be between 2,000 to 9,500 ppm or more for effective antimicrobial effectiveness.

The apparatus may further include a syringe 32 having a needle 34 for dispensing the ozonated fluid for medicinal use with ozonated aqueous solution being delivered by via a plunger 46 in a conventional maimer.

The plunger 46 further provides a means for charging the syringe 32 with ozonated fluid 22 upon insertion of the needle into the container 18 as indicated by the arrow 50.

Alternative embodiment 56 in accordance with the present invention is illustrated in FIG. 2 with common reference characters indicating identical or substantially similar structure shown in FIG. 1.

In this embodiment 56, the ozone generator 14 provides ozone for ozonating aqueous medium in the syringe 64 through an inlet 66. The syringe also includes an ozone detecting ink 68 similar to the ink, or sensor, 28 described in connection with the embodiment 10 shown in FIGS. 1 and 2.

Although there has been hereinabove described a specific apparatus for ozonation of fluids in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any clement which is not specifically disclosed herein. Accordingly, any and all modifications, variations or equivalent arrangements which may occur to those skilled in the art, should be considered to be within the scope of the present invention as defined in the appended claims.

What is claimed is:

1. Apparatus for ozonation of fluids, the apparatus comprising:
a source of ozone;
a fluid medium;
a container for receiving said fluid medium;
means for charging said fluid medium in said container with ozone; and
means for determining ozone content in the ozonated fluid medium within said container.

2. The apparatus according to claim 1 wherein the means for determining ozone control comprises an ozone detecting ink disposed in said container.

3. The apparatus according to claim 2 wherein the means for charging said liquid medium comprises a charging line connected to said source of ozone.

4. The apparatus according to claim 3 wherein said container comprises a syringe.

5. The apparatus according to claim 2 wherein said fluid medium comprises an oil.

6. The apparatus according to claim 2 wherein said fluid medium comprises a gas.

7. A method for ozonation of a fluid comprising;
providing a source of ozone;
providing a container;
disposing a fluid in said container;
interconnecting said container with said source of ozone to transfer ozone into said liquid; and
determining ozone contact in said fluid using an ozone detecting ink.
